(19) United States
(12) Patent Application Publication
(10) Pub. No.: US 2026/0337129 A1
(43) Pub. Date: Oct. 29, 2026

(54) METHOD FOR PRODUCING C2 HYDROCARBON COMPOUNDS AND AROMATIC COMPOUNDS FROM METHANE

(71) Applicant: KOREA RESEARCH INSTITUTE OF CHEMICAL TECHNOLOGY, Daejeon (KR)

(72) Inventors: Yong Tae KIM, Daejeon (KR); Jin Ju LEE, Daejeon (KR); Sung Woo LEE, Daejeon (KR); Jung Ho SHIN, Daejeon (KR); Seung Ju HAN, Daejeon (KR); Tsegay Gebrekidan GEBREYOHANNES, Daejeon (KR)

(73) Assignee: KOREA RESEARCH INSTITUTE OF CHEMICAL TECHNOLOGY, Daejeon (KR)

(21) Appl. No.: 19/418,282

(22) Filed: Dec. 12, 2025

Related U.S. Application Data
(63) Continuation of application No. PCT/KR2024/008017, filed on Jun. 12, 2024.

(30) Foreign Application Priority Data
Jul. 26, 2023 (KR) 10-2023-0097681

(51) Int. Cl. C07C 2/84

(52) U.S. Cl. CPC C07C 2/84; C07C 2523/28; C07C 2523/42; C07C 2523/745; C07C 2523/755

(57) ABSTRACT

The present invention relates to a method for producing C₂ hydrocarbons and aromatic compounds from methane, and more particularly, to a method for producing C₂ hydrocarbons and aromatic compounds from methane, wherein the method significantly improves methane consumption, provides high yields of C₂ hydrocarbons and aromatic compounds, and enhances catalyst regeneration efficiency, thereby providing an economically improved method for producing C₂ hydrocarbons and aromatic compounds from methane.

### CROSS REFERENCE TO RELATED APPLICATION

The present application is a continuation of PCT International Application No. PCT/KR2024/008017, which has an International filing date of Jun. 12, 2024, and which claims priority to Korean Patent Application No. 10-2023-0097681, filed on Jul. 26, 2023, the entire contents of each of which is incorporated herein for all purposes by this reference.

### TECHNICAL FIELD

The present invention relates to a method for producing C2 hydrocarbon compounds and aromatic compounds from methane, and more particularly, to a method for efficiently producing C2 hydrocarbon compounds and aromatic compounds from methane through a dehydroaromatization reaction and a non-oxidative direct conversion reaction of methane.

### BACKGROUND ART

In recent years, continuous efforts have been made to convert methane (CH₄), which can be obtained from natural gas, shale gas, and the like, into high value-added products such as transportation fuels or chemical raw materials. Representative examples of high value-added products obtainable from methane include olefins (such as ethylene, propylene, and butylene) and aromatic compounds. Technologies considered the most feasible include MTO (Methanol to Olefins), in which synthesis gas (H₂+CO) obtained through methane reforming is converted into light olefins via methanol, and FTO (Fischer-Tropsch to Olefins), in which light olefins are directly produced from synthesis gas.

However, in such technologies where high value-added products are produced via synthesis gas, hydrogen (H₂) or carbon monoxide (CO) is additionally required to remove oxygen atoms from carbon dioxide (CO₂), resulting in a decrease in the utilization efficiency of hydrogen atoms or carbon atoms in the overall process.

Therefore, there is a need for a new technology capable of directly converting methane into high value-added products without passing through synthesis gas. In order to directly convert methane into high value-added products, it is necessary to cleave the strongly bonded C—H bond (434 kJ/mol) in methane first to activate the methane. From this perspective, research has been actively conducted on the technology of oxidative coupling of methane (OCM), in which methane is activated using oxygen. However, in the OCM reaction as well, due to the violent reactivity of O₂, a large amount of thermodynamically stable H₂O and CO₂ is formed, and this has still been pointed out as a problem leading to a decrease in the utilization efficiency of hydrogen atoms or carbon atoms.

In order to solve such problems, technologies for producing ethylene, aromatic compounds, and the like by direct conversion of methane under strictly anaerobic or oxygen-free conditions have been developed. However, due to the low reactivity of methane, these processes are carried out at high temperature and high pressure, and the development of reactors and catalysts suitable for such conditions is essential. Nevertheless, according to research results to date, under such high-temperature and high-pressure conditions, rapid catalyst deactivation caused by carbon (coke) deposition on the catalyst has emerged as a critical issue (see Non-Patent Documents 0001 and 0002).

Accordingly, U.S. Pat. No. 4,424,401 discloses a method of aromatizing a hydrocarbon mixture by diluting acetylene with an inert gas, water, hydrogen, methane, and alcohol in the presence of a zeolite catalyst, ZSM-5. U.S. Pat. No. 8,013,196 discloses a method of producing ethylene by thermally converting a methane-containing feed into an acetylene-containing effluent through pyrolysis and subsequently hydrogenating the converted acetylene-containing effluent.

However, as in these prior arts, the methods of aromatizing methane or acetylene over zeolites or other catalysts also suffered from problems in that the performance of the catalyst appeared only for a very short time and then rapidly deactivated due to accumulation of coke fragments and rapid polymerization of acetylene. In addition, high contents of other by-products derived from acetylene conversion were formed.

In particular, coking, i.e., coke formation, has an undesirable effect on mass balance and yield, since the molecules of reactants and intermediates converted into coke can no longer be used for the desired reactions to provide aromatics. The coke selectivity obtained so far exceeds 20% in most cases, based on the converted aliphatic materials. Furthermore, coking degrades the activity of the catalyst within a relatively short time, thereby shortening the production cycle and necessitating catalyst regeneration. Coking also irreversibly shortens the lifespan of the catalyst.

Therefore, in the art, there is a need for the development of improved methods that enable more efficient and stable production of aromatic compounds and C2 hydrocarbons from methane.

### CITATION LIST

### Patent Literature

(Patent Document 1) U.S. Pat. No. 4,424,401 (Date of Registration: Jan. 3, 1984)
(Patent Document 2) U.S. Pat. No. 8,013,196 (Date of Registration: Sep. 6, 2011)

### Non-Patent Literature

(Non-Patent Document 1) X, Guo et al., Direct, Nonoxidative Conversion of Methane to Ethylene, Aromatics, and Hydrogen, Science, 344, 2014, 616~619
(Non-Patent Document 2) Mann Sakbodin et al., Hydrogen-Permeable Tubular Membrane Reactor: Promoting Conversion and Product Selectivity for Nonoxidative Activation of Methane over an FeVSiO2 Catalyst, Angew. Chem. 2016, 128, 16383-16386

### DISCLOSURE

### Technical Problem

The main object of the present invention is to solve the above-described problems and to provide a method for producing C2 hydrocarbon compounds and aromatic compounds from methane, which can significantly improve methane consumption, provide high yields of C2 hydrocarbon compounds and aromatic compounds, and enhance catalyst regeneration efficiency, thereby offering improved economics in the production of C2 hydrocarbon compounds and aromatic compounds from methane.

### Technical Solution

In order to achieve the above object, one embodiment of the present invention provides a method for producing C2 hydrocarbon compounds and aromatic compounds from methane, the method comprising:
(a) supplying a methane-containing feed to a reactor packed with a catalyst for dehydroaromatization reaction to produce unsaturated hydrocarbons containing aromatic compounds and unreacted materials;
(b) reacting the produced unsaturated hydrocarbons containing aromatic compounds and unreacted materials at 900 °C to 1,200 °C to produce hydrogen, C2 hydrocarbon compounds, and aromatic compounds;
(c) after step (b), regenerating the deactivated catalyst for dehydroaromatization reaction by supplying a hydrogen-containing gas thereto; and
(d) after step (c), sequentially repeating step (a) and step (b).

In a preferred embodiment of the present invention, the catalyst for dehydroaromatization reaction can comprise: a zeolite; and at least one transition metal selected from the group consisting of transition metals of the 6th period of the periodic table and transition metals of Groups 4 to 6 of the periodic table, the transition metal being supported on the zeolite.

In a preferred embodiment of the present invention, the transition metal can comprise at least one selected from the group consisting of molybdenum, nickel, iron, and platinum.

In a preferred embodiment of the present invention, the method for producing C2 hydrocarbon compounds and aromatic compounds from methane can be carried out stepwise in a single reactor.

In a preferred embodiment of the present invention, the reaction of step (a) can be carried out at 600 °C to 800 °C.

In a preferred embodiment of the present invention, the regeneration temperature of step (c) can be maintained the same as the reaction temperature of step (a).

In a preferred embodiment of the present invention, the hydrogen-containing gas of step (c) can contain hydrogen in an amount of 10 mol % to 100 mol %.

In a preferred embodiment of the present invention, after step (d), steps (c) and (d) can be repeatedly performed.

In a preferred embodiment of the present invention, the C2 hydrocarbon compound can comprise at least one selected from the group consisting of ethane, ethylene, and acetylene.

In a preferred embodiment of the present invention, after step (b), the method can further comprise a step of separately separating the hydrogen, C2 hydrocarbon compounds, and aromatic compounds produced in step (b).

In a preferred embodiment of the present invention, the separated hydrogen can be used as the hydrogen-containing gas in step (c).

### Advantageous Effects

According to the present invention, in the production of C2 hydrocarbon compounds and aromatic compounds from methane, methane consumption can be significantly improved, high yields of C2 hydrocarbons and aromatic compounds can be provided, and catalyst regeneration efficiency can be enhanced, thereby providing a method for producing C2 hydrocarbon compounds and aromatic compounds with improved economic efficiency.

### BRIEF DESCRIPTION OF DRAWINGS

FIG. 1 is a schematic diagram of a reactor for producing C2 hydrocarbon compounds and aromatic compounds according to one embodiment of the present invention.

FIG. 2 is a schematic diagram of a reactor for producing C2 hydrocarbon compounds and aromatic compounds according to another embodiment of the present invention.

FIG. 3 is a graph showing the results of measuring (a) methane conversion, (b) C2 hydrocarbon selectivity, (c) C3-C5 selectivity, and (d) aromatic compound selectivity according to the methane feed amount in Example 1 according to the present invention.

FIG. 4 is a graph showing the results of measuring (a) methane conversion amount, (b) C2 hydrocarbon production amount, (c) C3-C5 production amount, and (d) aromatic compound production amount according to the methane feed amount in Example 1 and Comparative Examples 1 to 4 according to the present invention.

FIG. 5 is a graph showing the results of measuring (a) methane conversion amount, (b) C2 hydrocarbon production amount, (c) C3-C5 production amount, and (d) aromatic compound production amount according to the methane feed amount in Comparative Examples 5 to 10 according to the present invention.

### DESCRIPTION OF SYMBOLS

100: Reactor
110: Inlet
111: Catalyst for dehydroaromatization reaction
121: Preheating section
122: Reaction zone section
130: Outlet

### MODE FOR DISCLOSURE

Unless defined otherwise, all technical and scientific terms used in the present specification have the same meaning as commonly understood by those skilled in the art to which the present invention pertains. In general, the nomenclature used in the present specification is well known in the art and commonly employed.

Throughout this specification, when any portion is referred to as “comprising” a certain component, it does not mean excluding other components unless otherwise stated, but rather means that other components can be further included. The term “reaction zone” means the space within the reactor in which reactants undergo a reaction.

The terms “comprising,” “including,” or “having,” as used in the present specification, indicate the presence of specified features, numbers, steps, operations, components, parts, or combinations thereof, but do not preclude the presence or addition of other features, numbers, steps, operations, components, parts, or combinations thereof not mentioned.

The following reactions and mixing, unless otherwise specified, can be carried out at room temperature and atmospheric pressure, and can be performed under conventional reaction and mixing conditions without any additional steps. However, interpretation should not deviate from what is clear to those skilled in the art.

The present invention provides a method for producing C2 hydrocarbon compounds and aromatic compounds from methane, the method comprising:
(a) supplying a methane-containing feed to a reactor packed with a catalyst for dehydroaromatization reaction to produce unsaturated hydrocarbons containing aromatic compounds and unreacted materials;
(b) reacting the produced unsaturated hydrocarbons containing aromatic compounds and unreacted materials at 900 °C to 1,200 °C to produce hydrogen, C2 hydrocarbon compounds, and aromatic compounds;
(c) after step (b), regenerating the deactivated catalyst for dehydroaromatization reaction by supplying a hydrogen-containing gas thereto; and
(d) after step (c), sequentially repeating step (a) and step (b).

More specifically, since the non-oxidative direct conversion reaction of methane proceeds at high temperature due to the low reactivity of methane, there is a problem in that catalyst activity is rapidly degraded by carbon (coke) deposition on the catalyst, thereby resulting in low methane conversion and low yields of C2 hydrocarbons and aromatic compounds.

Accordingly, in the present invention, prior to the non-oxidative direct conversion reaction of methane, a dehydroaromatization reaction of methane is carried out at a lower temperature than the non-oxidative direct conversion reaction to produce unsaturated hydrocarbons containing aromatic compounds, and the unsaturated hydrocarbons containing aromatic compounds, which act as hydrogen radical donors generated in the dehydroaromatization reaction, are used as a catalyst for activation of C—H bonds of methane in the non-oxidative direct conversion reaction. As a result, methane consumption can be greatly improved and the performance of the methane non-oxidative direct conversion reactor can be enhanced. Such linkage of non-oxidative methane conversion reactions allows the reaction to proceed more spontaneously and reduces the required energy, thereby improving the efficiency of the non-oxidative direct conversion of methane.

In addition, in the dehydroaromatization reaction of methane, the catalyst is regenerated within at least the same temperature range the dehydroaromatization reaction as temperature range by using a hydrogen-containing gas, whereby additional energy supply for catalyst regeneration is unnecessary, resulting in improved efficiency in terms of energy and process, and the methane conversion and yields of C2 hydrocarbons and aromatic compounds after catalyst regeneration can be improved.

Hereinafter, the present invention will be described in detail.

The method for producing C2 hydrocarbon compounds and aromatic compounds from methane according to the present invention first comprises supplying a methane-containing feed to a reactor packed with a catalyst for dehydroaromatization reaction to produce unsaturated hydrocarbons containing aromatic compounds and unreacted materials [(step (a))].

The methane-containing feed can be any mixture containing methane without limitation, and can include, for example, shale gas, natural gas, and the like. Preferably, in addition to methane, the feed can further contain an inert gas and/or a non-inert gas.

The methane contained in the methane-containing feed can be at least 2% (v/v) of the total volume of the methane-containing feed supplied into the reactor, and more preferably can be 40% (v/v) to 100% (v/v). The inert gas and/or non-inert gas can be 98% (v/v) or less, and more preferably 60% (v/v) or less, based on the total volume of the methane-containing feed.

The inert gas and/or non-inert gas serves to stably generate and maintain the reaction state. The inert gas can be nitrogen, helium, neon, argon, or krypton, and the non-inert gas can be carbon monoxide, hydrogen, carbon dioxide, water, ethane, ethylene, propane, propylene, butane, butylene, monohydric alcohols (C1-C5), dihydric alcohols (C2-C5), or alkanes (C2-C8). Preferably, the inert gas and the non-inert gas can be argon, nitrogen, hydrogen, oxygen, or water.

The methane-containing feed is supplied to a reactor packed with a catalyst for dehydroaromatization reaction, thereby producing a reaction product containing unsaturated hydrocarbons with aromatic compounds, unreacted materials, and hydrogen through the dehydroaromatization reaction.

The dehydroaromatization reaction of the methane-containing feed can produce aromatic compounds and hydrogen as shown in Reaction Formula 1 below. Reaction Formula 1 is provided to facilitate the explanation of the dehydroaromatization reaction of methane. The aromatic compounds generated in the dehydroaromatization reaction can include, in addition to benzene, toluene, ethylbenzene, styrene, xylene, and naphthalene. Unreacted methane, as well as ethane, ethylene, propane, propylene, butane, and butylene, can also be produced.

6CH₄→C₆H₆+9H₂ [Reaction Formula 1]

In this case, the dehydroaromatization reaction can be carried out at 600 °C to 800 °C, preferably 650 °C to 750 °C, and at 0.1 bar to 10 bar, preferably 0.1 bar to 5 bar.

The above reaction condition range is determined in consideration of the methane conversion rate and the yield of aromatic compounds, and has the advantage of maximizing the selectivity of methane to aromatic compounds. That is, under these conditions, the formation of paraffins, olefins, and coke is minimized, thereby supplying the concentration of hydrogen radical donors necessary for additional methane activation in the subsequent non-oxidative direct conversion reaction, while at the same time minimizing pressure buildup caused by coke formation during the reaction and minimizing carbon inefficiency due to coke formation.

In the dehydroaromatization reaction, when the reaction temperature is less than 600 °C, the catalytic activity may be too low, resulting in too low a yield of aromatic compounds. When the temperature exceeds 800 °C, the deactivation rate of the dehydroaromatization catalyst due to coke formation becomes rapid, and therefore the residence time of methane in the reactor must be minimized to suppress coke formation, and a large amount of energy is required for reactor heating, which can cause problems.

In addition, in the dehydroaromatization reaction, when the reaction pressure is less than 1 bar, coke formation can be suppressed, but the energy efficiency is low due to low methane activation. When the pressure exceeds 10 bar, coke formation is promoted, and therefore reactor residence time and product cooling must be efficiently designed, which can cause problems.

In the dehydroaromatization reaction, the space velocity can be 2,000 ml h⁻¹ gcat⁻¹ to 12,000 ml h⁻¹ gcat⁻¹. When the space velocity in the dehydroaromatization reaction satisfies this range, the chain reactivity from methane to ethane and ethylene, and from the ethane and ethylene to aromatic compounds, can be controlled so as to maximize the formation of aromatic compounds, while also minimizing coke formation from aromatic compounds due to excessive reaction.

Here, the space velocity (Weight Hourly Space Velocity: WHSV) refers to the flow rate of the reaction gas per reactor volume in the dehydroaromatization reaction zone, and can be calculated as the gas volume flow rate (ml/hr) in the dehydroaromatization reaction zone divided by the catalyst weight (g).

Meanwhile, in the present invention, the catalyst for dehydroaromatization reaction can include a catalyst capable of promoting the dehydroaromatization reaction. The catalyst for dehydroaromatization reaction may be one in which a catalytically active component is supported on a catalyst support that provides acid sites to induce the dehydroaromatization reaction.

The catalyst support providing the acid sites can be a metal silicate, preferably a zeolite, and more preferably a zeolite having an MFI structure such as Silicalite-1, ZSM-5, or TS-1. In this case, the zeolite can contain, in addition to Al, additional elements of Group III such as Ga, B, or In. In addition, non-limiting examples of dopants can include noble metals such as Rh, Pd, Ag, Ir, Pt, and/or Au, or transition metals such as Fe, Ni, Co, and/or Cu.

Furthermore, the SiO₂/Al₂O₃ molar ratio of the zeolite can be 1 to 300, preferably 10 to 100, and more preferably 10 to 70. When zeolites within this range are used, the concentration of Brønsted acid sites can be increased relative to the concentration of Lewis acid sites, thereby providing acid sites that selectively convert ethane and ethylene compounds into aromatic compounds, while also enhancing the dispersion of the catalytically active components on the surface of the zeolite.

Meanwhile, non-limiting examples of the catalytically active component can include at least one transition metal selected from the group consisting of transition metals of the 6th period of the periodic table and transition metals of Groups 4 to 6 of the periodic table, such as molybdenum, nickel, iron, and platinum. Preferably, the catalytically active component can include molybdenum (Mo). The molybdenum can be supported with high dispersion while neutralizing strong Brønsted acid sites of the catalyst support during catalyst preparation, and can be activated into molybdenum carbide during the reaction, thereby converting methane into ethane and ethylene through initial activation of methane. Accordingly, sufficient molybdenum active sites capable of interacting with the acid sites of the catalyst support must be present in order to exhibit reactivity.

The loading of the catalytically active component, i.e., the transition metal, can be 1 wt % to 8 wt % based on the total weight of the catalyst, and preferably 4 wt % to 7 wt %. Within this range, the catalyst for the dehydroaromatization reaction can maximize surface active sites for the initial activation of methane while suppressing the formation of coke containing polyaromatic compounds at the acid sites, thereby increasing the selectivity for aromatic compounds.

As described above, the methane-containing feed undergoes a dehydroaromatization reaction to produce aromatic compounds, unsaturated hydrocarbons containing aromatic compounds that act as hydrogen radical donors, and unreacted materials. The generated aromatic compounds, unsaturated hydrocarbons containing aromatic compounds that act as hydrogen radical donors, and unreacted materials are then subjected to a non-oxidative direct conversion reaction, wherein methane is activated as shown in Reaction Formula 2 below to synthesize hydrogen, C2 hydrocarbon compounds (ethylene, acetylene, and ethane), and aromatic compounds [(step (b))]. At this time, the aromatic compounds reversibly donate and accept hydrogen radicals, thereby promoting the initial activation of methane.

CH₄+C₆H₆→CH₃·+C₆H₅·+H₂

2CH₃·→C₂H₆

C₂H₆→C₂H₄+H₂

C₂H₄→C₂H₂+2H₂

3C₂H₂→C₆H₆

2C₆H₅·+H₂→2C₂H₆

[Reaction Formula 2]

The non-oxidative direct conversion reaction can be carried out at 900 °C to 1,200 °C, preferably 950 °C to 1, 150 °C, and at 0.1 bar to 10 bar, preferably 0.1 bar to 5 bar. The space velocity in the reaction zone may be 200 h⁻¹ to 12,000 h⁻¹.

The above reaction conditions are determined in consideration of the selectivity and yield of hydrocarbons, and have the advantage of maximizing the selectivity of methane to hydrocarbons. That is, under these conditions, coke formation is minimized, thereby minimizing pressure drop caused by coke formation during the reaction and carbon deposition due to coke formation.

In the non-oxidative direct conversion reaction, when the reaction temperature is less than 900 °C, the reactivity is low, which may result in the need for a larger reactor corresponding to the reaction zone of the non-oxidative direct conversion reaction. When the temperature exceeds 1, 200 °C, side reactions (dehydrogenation and coupling) are promoted, which may consequently promote coke formation.

In addition, in the non-oxidative direct conversion reaction, when the reaction pressure is less than 0.1 bar, the reactivity is low, which may result in the need for a larger reactor corresponding to the reaction zone of the non-oxidative direct conversion reaction. When the pressure exceeds 10 bar, side reactions (dehydrogenation and coupling) become dominant, which may consequently promote coke formation.

Such a non-oxidative direct conversion reaction may have a ratio of the space velocity (GHSV) of the non-oxidative direct conversion reaction to the space velocity (WHSV) of the dehydroaromatization catalyst as 0.01 g ml⁻¹ to 6 g ml⁻¹. When the ratio of the space velocity (GHSV) of the non-oxidative direct conversion reaction to the space velocity (WHSV) of the dehydroaromatization catalyst is less than 0.01 g ml⁻¹, the linkage effect between the non-oxidative direct conversion reaction and the dehydroaromatization reaction is reduced, which may lower the efficiency of the non-oxidative direct conversion reactor. When the ratio exceeds 6 g ml⁻¹, the reaction temperature of the non-oxidative direct conversion reaction must be increased compared to that of the dehydroaromatization reaction, thereby causing a problem of decreased thermal efficiency.

The dehydroaromatization reaction and the non-oxidative direct conversion reaction according to the present invention can be sequentially carried out in a single reactor or in two or more reactors. From the viewpoint of optimizing heat exchange due to reaction heat and temperature gradients, it is preferable to carry out the reaction in a single reactor, and more preferably in a single reactor having a double-tube structure or a radial direction.

As one embodiment, when the dehydroaromatization reaction and the non-oxidative direct conversion reaction are performed in a single reactor, the reactor (100) can be divided into a preheating section (121) and a reaction zone (122), as shown in FIG. 1. The preheating section (121) thus divided is packed with the dehydroaromatization catalyst (111), and the methane-containing feed supplied in the presence of the packed dehydroaromatization catalyst is reacted to generate unsaturated hydrocarbons containing aromatic compounds and unreacted materials. The generated unsaturated hydrocarbons containing aromatic compounds and unreacted materials are then introduced into the reaction zone (122), where the non-oxidative direct conversion reaction of the unsaturated hydrocarbons containing aromatic compounds and unreacted materials produces hydrogen, C2 hydrocarbons, and aromatic compounds.

At this time, the reactor can be any reactor known in the technical field to which the present invention belongs, and its dimensions or shape can vary depending on production capacity, feed amount, and catalyst, and it can be adjusted by various methods known to those skilled in the art. Structurally, however, the reactor may be a tubular reactor in which an inlet (110) is formed on one side to introduce the methane-containing feed, the introduced methane-containing feed undergoes reaction in the reaction zone, and an outlet (130) is formed on the opposite side of the inlet to discharge the reacted product to the outside or downstream.

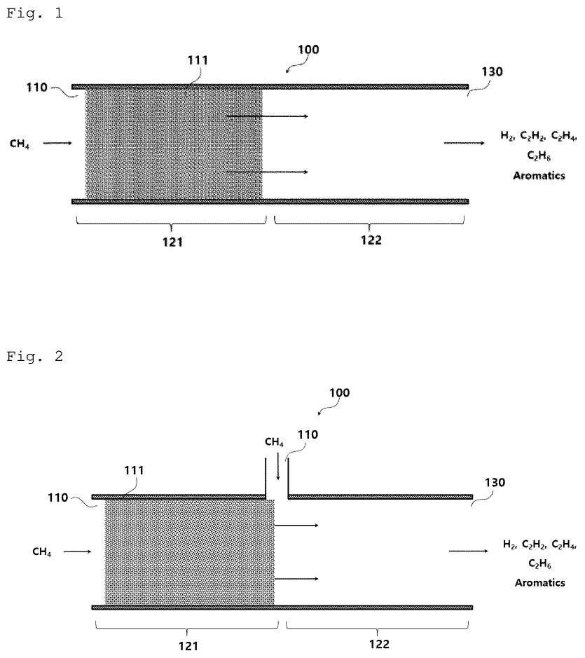

In addition, the position of the inlet of the reactor can be arranged at the top, bottom, right, or left of the reactor without limitation, and a plurality of inlets can be disposed. Preferably, in order to maximize reactivity while lowering the concentration of radical donors generated in the dehydroaromatization reaction and suppressing coke formation, as shown in FIG. 2, an additional inlet (110) for supplying a methane-containing feed can be disposed between the reaction zone of the dehydroaromatization reaction (preheating section, 121) and the reaction zone of the non-oxidative direct conversion reaction (reaction zone, 122).

The hydrogen, C2 hydrocarbons, and aromatic compounds thus generated can be discharged to the outside or discharged downstream. At this time, the C2 hydrocarbons can include ethane, ethylene, and acetylene, and the aromatic compounds can include aromatic compounds such as benzene, toluene, xylene, ethylbenzene, and naphthalene.

The hydrogen, C2 hydrocarbons, and aromatic compounds discharged downstream may further undergo a step of being separated individually. The separation of the hydrogen, C2 hydrocarbons, and aromatic compounds can be carried out by means of devices or methods known in the technical field to which the present invention belongs, and the devices and separation methods can include distillation, membranes, and solvent extraction. The hydrogen separated from the C2 hydrocarbons and aromatic compounds can be used as the hydrogen-containing gas for catalyst regeneration, which will be described later.

After the dehydroaromatization reaction and the non-oxidative direct conversion reaction, the deactivated dehydroaromatization catalyst is regenerated using a hydrogen-containing gas [(c) step].

In this case, the hydrogen-containing gas can be any non-oxidizing gas containing hydrogen without limitation, and preferably can contain, in addition to hydrogen, an inert gas such as argon or nitrogen, and more preferably, the hydrogen content can be 10 mol % to 100 mol %.

If, in the hydrogen-containing gas, the hydrogen content is less than 10 mol %, a long time is required to remove coke deposited on the catalyst surface, which may result in low energy efficiency. If the hydrogen content exceeds 100 mol %, the catalyst regeneration time can be shortened; however, design of reaction conditions capable of controlling the heat generated during regeneration may be required. Such reaction conditions requiring design may include reaction flow rate, reaction time, and the like.

By using a hydrogen-containing gas as the regeneration gas during catalyst regeneration in this manner, not only can the chemical stability of transition metal carbides (such as molybdenum, etc.), which are the metallic active sites of the dehydroaromatization reaction catalyst, be increased during regeneration, but also the regeneration heat can be minimized to reduce structural deformation of catalyst supports such as zeolites. For example, if regeneration is attempted using air or oxygen, irreversible damage to the above-mentioned transition metals and catalyst supports may occur; however, when a hydrogen-containing gas is used, such effects can be selectively controlled.

In addition, before and/or after the regeneration step of regenerating the catalyst, purging can be carried out with an inert gas; however, when hydrogen is used as the regeneration gas, it is not essential from a process standpoint. The purge gas can include inert gases, steam, or air.

In the regeneration step, the catalyst regeneration temperature can be maintained within the same temperature range as the above-described dehydroaromatization reaction temperature. Accordingly, in the production of C2 hydrocarbon compounds and aromatic compounds according to the present invention, since additional energy supply is not required during catalyst regeneration, improved efficiency in terms of energy and process can be achieved.

If the catalyst regeneration temperature is lower than the dehydroaromatization reaction temperature range, not only will the time required to regenerate the catalyst be prolonged, but only amorphous coke can be selectively removed, and deactivation of the catalyst due to increased deposition of crystalline coke cannot be prevented. On the other hand, if the regeneration temperature exceeds the dehydroaromatization reaction temperature range, local temperature rise caused by coke oxidation and structural changes in catalyst supports such as zeolites may occur, resulting in hotspot formation and catalyst sintering. Consequently, this may further deactivate the catalyst, leading to losses in terms of activity.

Meanwhile, the catalyst regeneration time can be adjusted depending on the amount of catalyst, regeneration conditions, and the like, and in terms of energy efficiency, it is preferably 0.5 to 3 times, more preferably 3 times, relative to the reaction time.

After the regeneration step of the dehydroaromatization catalyst, step (d) is performed by resupplying a methane-containing feed to the regenerated catalyst and sequentially repeating the dehydroaromatization reaction of step (a) and the non-oxidative direct conversion reaction of step (b).

In addition, in the method for producing C2 hydrocarbon compounds and aromatic compounds according to the present invention, after step (d), the deactivated catalyst can [(step (c))], followed by repeating the be regenerated dehydroaromatization reaction and the non-oxidative direct conversion reaction [(step (d))].

In this case, when producing C2 hydrocarbon compounds and aromatic compounds according to the manufacturing method of the present invention, since the set temperature for producing the C2 hydrocarbon compounds and aromatic compounds is the same as the temperature for regenerating the catalyst, regeneration can be performed by injecting a hydrogen-containing gas without supplying additional heat sources. Thus, process energy efficiency is excellent, and the heating and cooling processes during reaction and regeneration can be omitted, thereby improving ease of process operation, while the purge process of the reactor during heating and cooling can also be omitted, reducing the use of purge gas.

Hereinafter, the present invention will be described in more detail through specific examples. The following examples are merely illustrative to aid in understanding the present invention, and the scope of the present invention is not limited thereto.

### Preparation Example 1: Preparation of Dehydroaromatization Catalyst

A 6 wt % Mo/H-ZSM-5 (SiO₂/Al₂O₃=30) catalyst was prepared by a wet impregnation method. 1.15 g of ammonium heptamolybdate [(NH₄)₆Mo₇O₂₄·4H₂O], used as the Mo precursor, was dissolved in 20 g of deionized water (D.I. water). To this solution, 10 g of H-ZSM-5 (SiO₂/Al₂O₃=30) was added and maintained at 40 °C for 30 minutes. Thereafter, water was removed at 50 °C using a rotary vacuum evaporator (RV 10 control, IKA) to obtain a solid. The obtained solid was dried at 110 °C for 10 hours and then calcined in an air atmosphere at 500 °C to prepare the dehydroaromatization catalyst.

### Example 1

Using the reactor shown in FIG. 1, C₂ hydrocarbons and aromatic compounds were produced from methane. The reactor was a fixed-bed reactor, in which a quartz tubular reactor with an inner diameter of 4 mm was installed in a furnace having two separate heating zones of 10 cm and 10 cm in height, respectively. The upper part was designated as the preheating section (reaction zone for dehydroaromatization), and the lower part was designated as the reaction section (reaction zone for non-oxidative direct conversion). The catalyst prepared in Preparation Example 1 was crushed to a particle size of 425 μm to 850 μm, and 0.20 g of the catalyst was packed into the preheating section (the reaction zone for dehydroaromatization). The methane-containing feed [90% (v/v) CH₄, 10% (v/v) Ar] was supplied into the reactor at a flow rate of 20 ml/min using mass flow controllers. The preheating section was maintained at 700 °C, and the reaction section was maintained at 1,020 °C, with the reaction carried out for 15 minutes (Table 1). Argon (Ar) gas was used as an internal standard. Subsequently, catalyst regeneration was performed as shown in Table 2, by switching the reaction gas to 100% H₂ and supplying it at 20 ml/min for 45 minutes. This example was conducted periodically over a total of 50 hours.

### Comparative Example 1

C₂ hydrocarbons and aromatic compounds were produced in the same manner as in Example 1, except that, as shown in Table 2, 0.5% O₂ was used as the gas for catalyst regeneration. After reacting the methane-containing feed [90% (v/v) CH₄, 10% (v/v) Ar] at 20 ml/min for 15 minutes, the reaction gas was switched to 0.5% O₂ and supplied at 20 ml/min for 45 minutes to regenerate the catalyst. This comparative example was carried out periodically for a total of 50 hours.

### Comparative Example 2

C₂ hydrocarbons and aromatic compounds were produced using the reactor shown in FIG. 1. The reactor was a fixed-bed reactor, in which a tubular quartz reactor with an internal diameter of 4 mm was installed in a furnace having separate heating zones, each 10 cm in height. The upper portion was designated as the preheating zone (reaction zone for dehydroaromatization), and the lower portion was designated as the reaction zone (reaction zone for dehydroaromatization). The catalyst prepared in Preparation Example 1 was crushed to a particle size of 425 μm to 850 μm, and 0.20 g was packed inside the preheating zone. The methane-containing feed [90% (v/v) CH₄, 10% (v/v) Ar] was supplied into the reactor at 20 ml/min using mass flow controllers, and the reaction was carried out for 15 minutes at 700 °C in the preheating zone and 700 °C in the reaction zone (Table 1). At this time, argon (Ar) gas was used as the internal standard. Thereafter, for catalyst regeneration, the reaction gas was switched to 100% H₂ and supplied at 20 ml/min for 45 minutes, as shown in Table 2. This experiment was periodically carried out for a total of 50 hours.

### Comparative Example 3

C₂ hydrocarbons and aromatic compounds were produced using the reactor shown in FIG. 1. The reactor was a fixed-bed reactor, in which a tubular quartz reactor with an internal diameter of 4 mm was installed in a furnace having separate heating zones, each 10 cm in height. The upper portion was designated as the preheating zone (reaction zone for dehydroaromatization), and the lower portion was designated as the reaction zone (reaction zone for non-oxidative direct conversion). The catalyst prepared in Preparation Example 1 was crushed to a particle size of 425 μm to 850 μm, and 0.20 g was packed inside the preheating zone. The methane-containing feed [90% (v/v) CH₄, 10% (v/v) Ar] was supplied into the reactor at 20 ml/min using mass flow controllers, and the reaction was carried out for 15 minutes at 700 °C in the preheating zone and 700 °C in the reaction zone (Table 1). At this time, argon (Ar) gas was used as the internal standard. Thereafter, for catalyst regeneration, the reaction gas was switched to 0.5% O₂ and supplied at 20 ml/min for 45 minutes, as shown in Table 2. This experiment was periodically carried out for a total of 50 hours.

### Comparative Example 4

C₂ hydrocarbons and aromatic compounds were produced using the reactor shown in FIG. 1. The reactor was a fixed-bed reactor, in which a tubular quartz reactor with an internal diameter of 4 mm was installed in a furnace having separate heating zones, each 10 cm in height. The upper portion was designated as the preheating zone (reaction for zone dehydroaromatization), and the lower portion was designated as the reaction zone (reaction for non-oxidative direct conversion). The catalyst prepared in Preparation Example 1 was crushed to a particle size of 425 μm to 850 μm, and 0.80 g was packed inside the preheating zone. The methane-containing feed [90% (v/v) CH₄, 10% (v/v) Ar] was supplied into the reactor at 20 ml/min using mass flow controllers, and the reaction was carried out for 15 minutes at 700 °C in the preheating zone and 700 °C in the reaction zone (Table 1). At this time, argon (Ar) gas was used as the internal standard. Thereafter, for catalyst regeneration, the reaction gas was switched to 100% H₂ and supplied at 20 ml/min for 45 minutes, as shown in Table 2. This comparative example was periodically carried out for a total of 10 hours.

### Comparative Example 5

C₂ hydrocarbons and aromatic compounds were produced using the reactor shown in FIG. 1. The reactor was a fixed-bed reactor, in which a tubular quartz reactor with an internal diameter of 4 mm was installed in a furnace having two separate heating zones, each 10 cm in height. The upper portion was designated as the preheating zone (reaction for zone dehydroaromatization), and the lower portion was designated as the reaction zone (reaction zone for non-oxidative direct conversion). The catalyst prepared in Preparation Example 1 was crushed to a particle size of 425 μm to 850 μm, and 0.20 g was packed inside the preheating zone. The methane-containing feed [90% (v/v) CH₄, 10% (v/v) Ar] was supplied into the reactor at 20 ml/min using mass flow controllers, and the reaction was carried out for 4 hours at 700 °C in the preheating zone and 700 °C in the reaction zone (Table 1). At this time, argon (Ar) gas was used as the internal standard. Thereafter, for catalyst regeneration, the reaction gas was switched to 10% O₂ and supplied at 20 ml/min for 30 minutes, as shown in Table 2. This experiment was periodically carried out for a total of 36 hours.

### Comparative Example 6

C₂ hydrocarbons and aromatic compounds were produced using the reactor shown in FIG. 1. The reactor was a fixed-bed reactor, in which a tubular quartz reactor with an internal diameter of 4 mm was installed in a furnace having two separate heating zones, each 10 cm in height. The upper portion was designated as the preheating zone (reaction zone for dehydroaromatization), and the lower portion was designated as the reaction zone (reaction zone for non-oxidative direct conversion). The catalyst prepared in Preparation Example 1 was crushed to a particle size of 425 μm to 850 μm, and 0.20 g was packed inside the preheating zone. The methane-containing feed [90% (v/v) CH₄, 10% (v/v) Ar] was supplied into the reactor at 20 ml/min using mass flow controllers, and the reaction was carried out for 4 hours at 700 °C in the preheating zone and 700 °C in the reaction zone (Table 1). At this time, argon (Ar) gas was used as the internal standard. Thereafter, for catalyst regeneration, the reaction gas was switched to 10% O₂ and supplied at 20 ml/min for 10 minutes, as shown in Table 2. This experiment was periodically carried out for a total of 36 hours.

### Comparative Example 7

C₂ hydrocarbons and aromatic compounds were produced using the reactor shown in FIG. 1. The reactor was a fixed-bed reactor, in which a tubular quartz reactor with an internal diameter of 4 mm was installed in a furnace having two separate heating zones, each 10 cm in height. The upper portion was designated as the preheating zone (reaction zone for dehydroaromatization), and the lower portion was designated as the reaction zone (reaction zone for non-oxidative direct conversion). The catalyst prepared in Preparation Example 1 was crushed to a particle size of 425 μm to 850 μm, and 0.20 g was packed inside the preheating zone. The methane-containing feed [90% (v/v) CH₄, 10% (v/v) Ar] was supplied into the reactor at 20 ml/min using mass flow controllers, and the reaction was carried out for 4 hours at 700 °C in the preheating zone and 700 °C in the reaction zone (Table 1). At this time, argon (Ar) gas was used as the internal standard. Thereafter, for catalyst regeneration, the reaction gas was switched to 0.5% O₂ and supplied at 20 ml/min for 10 minutes, as shown in Table 2. This experiment was periodically carried out for a total of 36 hours.

### Comparative Example 8

C₂ hydrocarbons and aromatic compounds were produced using the reactor shown in FIG. 1. The reactor was a fixed-bed reactor, in which a tubular quartz reactor with an internal diameter of 4 mm was installed in a furnace having two separate heating zones, each 10 cm in height. The upper portion was designated as the preheating zone (reaction zone for dehydroaromatization), and the lower portion was designated as the reaction zone (reaction zone for non-oxidative direct conversion). The catalyst prepared in Preparation Example 1 was crushed to a particle size of 425 μm to 850 μm, and 0.20 g was packed inside the preheating zone. The methane-containing feed [90% (v/v) CH₄, 10% (v/v) Ar] was supplied into the reactor at 20 ml/min using mass flow controllers, and the reaction was carried out for 4 hours at 700 °C in the preheating zone and 700 °C in the reaction zone (Table 1). At this time, argon (Ar) gas was used as the internal standard. Thereafter, for catalyst regeneration, the reaction gas was switched to 0.03% O₂ and supplied at 20 ml/min for 10 minutes, as shown in Table 2. This experiment was periodically carried out for a total of 36 hours.

### Comparative Example 9

C₂ hydrocarbons and aromatic compounds were produced using the reactor shown in FIG. 1. The reactor was a fixed-bed reactor, in which a tubular quartz reactor with an internal diameter of 4 mm was installed in a furnace having two separate heating zones, each 10 cm in height. The upper portion was designated as the preheating zone (reaction zone for dehydroaromatization), and the lower portion was designated as the reaction zone (reaction zone for non-oxidative direct conversion). The catalyst prepared in Preparation Example 1 was crushed to a particle size of 425 μm to 850 μm, and 0.20 g was packed inside the preheating zone. The methane-containing feed [90% (v/v) CH₄, 10% (v/v) Ar] was supplied into the reactor at 20 ml/min using mass flow controllers, and the reaction was carried out for 4 hours at 700 °C in the preheating zone and 700 °C in the reaction zone (Table 1). At this time, argon (Ar) gas was used as the internal standard. Thereafter, for catalyst regeneration, as shown in Table 2, the preheating zone was maintained at 500 °C and the reaction zone at 500 °C, and the reaction gas was switched to 0.5% O₂ and supplied at 20 ml/min for 30 minutes to regenerate the catalyst. This experiment was periodically carried out for a total of 36 hours.

### Comparative Example 10

C₂ hydrocarbons and aromatic compounds were produced using the reactor shown in FIG. 1. The reactor was a fixed-bed reactor, in which a tubular quartz reactor with an internal diameter of 4 mm was installed in a furnace having two separate heating zones, each 10 cm in height. The upper portion was designated as the preheating zone (reaction zone for dehydroaromatization), and the lower portion was designated as the reaction zone (reaction zone for non-oxidative direct conversion). The catalyst prepared in Preparation Example 1 was crushed to a particle size of 425 μm to 850 μm, and 0.20 g was packed inside the preheating zone. The methane-containing feed [90% (v/v) CH₄, 10% (v/v) Ar] was supplied into the reactor at 20 ml/min using mass flow controllers, and the reaction was carried out for 4 hours at 700 °C in the preheating zone and 700 °C in the reaction zone (Table 1). At this time, argon (Ar) gas was used as the internal standard. Thereafter, for catalyst regeneration, as shown in Table 2, the preheating zone was maintained at 500 °C and the reaction zone at 500 °C, and the reaction gas was switched to 0.03% O₂ and supplied at 20 ml/min for 30 minutes to regenerate the catalyst. This experiment was periodically carried out for a total of 36 hours.

TABLE 1

| | Reaction | | Space Velocity | | |
|---|---|---|---|---|---|
| | Temperature (°C) | | Preheating | Reaction | |
| | | Reaction | Section | Zone | Reaction |
| Classifi- | Preheating | Zone | (ml g_{cat}⁻¹ | Section | Time |
| cation | Section | Section | h⁻¹) | (h⁻¹) | (min) |
| Example 1 | 700 | 1020 | 6,000 | 955 | 15 |
| Comparative | 700 | 1020 | 6,000 | 955 | 15 |
| Example 1 | | | | | |
| Comparative | 700 | 700 | 6,000 | 955 | 15 |
| Example 2 | | | | | |
| Comparative | 700 | 700 | 6,000 | 955 | 15 |
| Example 3 | | | | | |
| Comparative | 700 | 700 | 1,500 | 955 | 15 |
| Example 4 | | | | | |
| Comparative | 700 | 700 | 6,000 | 955 | 240 |
| Example 5 | | | | | |
| Comparative | 700 | 700 | 6,000 | 955 | 240 |
| Example 6 | | | | | |
| Comparative | 700 | 700 | 6,000 | 955 | 240 |
| Example 7 | | | | | |
| Comparative | 700 | 700 | 6,000 | 955 | 240 |
| Example 8 | | | | | |
| Comparative | 700 | 700 | 6,000 | 955 | 240 |
| Example 9 | | | | | |
| Comparative | 700 | 700 | 6,000 | 955 | 240 |
| Example 10 | | | | | |

Preheating Section: Dehydroaromatization Reaction
Reaction Zone Section: Non-oxidative Direct Conversion Reaction

TABLE 2

| | | Regeneration | | Space Velocity | | | |
|---|---|---|---|---|---|---|---|
| | | Temperature (°C) | | Preheating | Reaction | | |
| | | | Reaction | Section | Zone | Regeneration | |
| | Regeneration | Preheating | Zone | (ml g_{cat}⁻¹ | Section | Time | Cycle |
| Classification | Gas | Section | Section | h⁻¹) | (h⁻¹) | (min) | (hr) |
| Example 1 | 100 | 700 | 1020 | 6,000 | 955 | 15 | 50 |
| | mol % H₂ | | | | | | |
| Comparative | 0.5 | 700 | 1020 | 6,000 | 955 | 15 | 50 |
| Example 1 | mol % O₂ | | | | | | |
| Comparative | 100 | 700 | 700 | 6,000 | 955 | 15 | 50 |
| Example 2 | mol % H₂ | | | | | | |
| Comparative | 0.5 | 700 | 700 | 6,000 | 955 | 15 | 50 |
| Example 3 | mol O₂ | | | | | | |
| Comparative | 100 | 700 | 700 | 1,500 | 955 | 15 | 10 |
| Example 4 | mol % H₂ | | | | | | |
| Comparative | 10 mol % | 700 | 700 | 6,000 | 955 | 240 | 36 |
| Example 5 | O₂ | | | | | | |
| Comparative | 10 mol % | 700 | 700 | 6,000 | 955 | 240 | 36 |
| Example 6 | O₂ | | | | | | |
| Comparative | 0.5 | 700 | 700 | 6,000 | 955 | 240 | 36 |
| Example 7 | mol % O₂ | | | | | | |
| Comparative | 0.03 | 700 | 700 | 6,000 | 955 | 240 | 36 |
| Example 8 | mol % O₂ | | | | | | |
| Comparative | 0.5 | 500 | 700 | 6,000 | 955 | 240 | 36 |
| Example 9 | mol % O₂ | | | | | | |
| Comparative | 0.03 | 500 | 700 | 6,000 | 955 | 240 | 36 |
| Example 10 | mol % O₂ | | | | | | |

Preheating Section: Dehydroaromatization Reaction
Reaction Zone Section: Non-oxidative Direct Conversion Reaction

### Experimental Example 1: Measurement of Methane Conversion Rate and Selectivity of Aromatic Compounds and C₂ Hydrocarbons

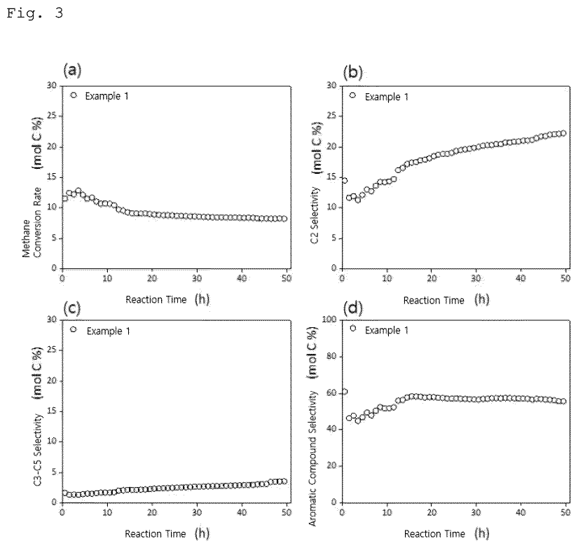

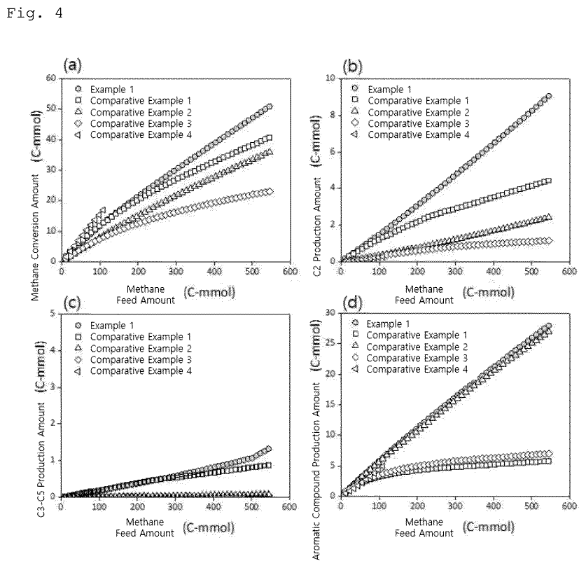

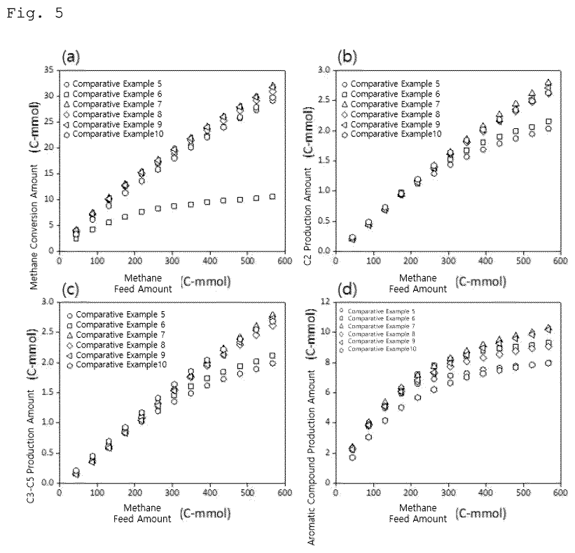

The products obtained in the Examples and Comparative Examples were analyzed every hour after 30 minutes from the start of reactant feeding using an Agilent 8890 GC connected online. The gaseous products were analyzed with a Thermal Conductivity Detector (TCD) connected to a ShinCarbon ST 80/100 column and two Flame Ionization Detectors (FID) connected respectively to an Rt-alumina BOND column and an RTx-VMS column. Hydrogen, methane, argon, and C₂ hydrocarbons (ethane, ethylene, and acetylene) were separated on the ShinCarbon ST 80/100 column and detected by the TCD, and the conversion rate was calculated based on the ratio of the peak area of methane to that of argon as the internal standard. Light hydrocarbons in the C₁ to C₅ range and benzene were separated on the Rt-alumina BOND column and detected by an FID, while aromatic compounds including benzene were separated on the RTx-VMS column and detected by an FID. All gases were quantified using standard samples. Methane conversion (C-mmol) and product yield (C-mmol) were integrated over the reaction time from the amounts detected at each interval and converted into values, as shown in FIGS. 3 to 5.

As shown in FIGS. 3 to 5, in Example 1, it was found that through optimization of the reaction and regeneration, the deactivation of methane conversion was minimized, while C₂ hydrocarbons including ethane, ethylene, and acetylene, as well as aromatic compounds including benzene, toluene, and naphthalene, could be stably produced. From this, it was confirmed that when a catalyst is used in the dehydroaromatization reaction and the non-oxidative direct conversion reaction is utilized as a zone to promote methane conversion, the periodic optimization of reaction and regeneration is suitable for the stable production of hydrocarbons from methane.

In particular, compared to Comparative Examples 1 to 4, Example 1 confirmed that performing the non-oxidative direct conversion reaction to promote pyrolysis exhibited higher reaction performance than carrying out only the dehydroaromatization reaction. Moreover, when hydrogen was used as the regeneration gas instead of oxygen in periodic catalyst regeneration, effective improvement in stability was achieved. Furthermore, through the comparison between Example 1 and Comparative Example 4, it was confirmed that when the concentration of aromatic compounds was abundant in the reaction zone (preheating zone) of the dehydroaromatization reaction, the C₂ yield in the reaction zone (reaction zone) of the non-oxidative direct conversion reaction was further enhanced.

It was also confirmed, through the comparison between Example 1 and Comparative Examples 5 to 10, that even when the periodic catalyst regeneration was optimized using oxygen as the regeneration gas, the methane conversion and hydrocarbon production according to the methane feed amount were lower than in the case where hydrogen was used as the regeneration gas.

Although the present invention has been described with reference to the foregoing examples and the accompanying drawings, various different embodiments can be devised within the concept and scope of the present invention. Therefore, the scope of the present invention should be determined by the appended claims and their equivalents, and is not limited by the specific embodiments described in this specification.

1. A method for producing C2 hydrocarbon compounds and aromatic compounds from methane, the method comprising:
(a) supplying a methane-containing feed to a reactor packed with a catalyst for dehydroaromatization reaction to produce unsaturated hydrocarbons containing aromatic compounds and unreacted materials;
(b) reacting the produced unsaturated hydrocarbons containing aromatic compounds and unreacted materials at 900 °C to 1,200 °C to produce hydrogen, C₂ hydrocarbon compounds, and aromatic compounds;
(c) after the step (b), regenerating the deactivated catalyst for dehydroaromatization reaction by supplying a hydrogen-containing gas thereto; and
(d) after the step (c), sequentially repeating step (a) and step (b).

2. The method for producing C2 hydrocarbon compounds and aromatic compounds from methane according to claim 1,
wherein the catalyst for dehydroaromatization reaction comprises:
a zeolite; and
at least one transition metal selected from the group consisting of transition metals of the 6th period of the periodic table and transition metals of Groups 4 to 6 of the periodic table, the transition metal being supported on the zeolite.

3. The method for producing C2 hydrocarbon compounds and aromatic compounds from methane according to claim 2,
wherein the transition metal comprises at least one selected from the group consisting of molybdenum, nickel, iron, and platinum.

4. The method for producing C2 hydrocarbon compounds and aromatic compounds from methane according to claim 1,
wherein the method for producing C2 hydrocarbon compounds and aromatic compounds from methane is carried out stepwise in a single reactor.

5. The method for producing C2 hydrocarbon compounds and aromatic compounds from methane according to claim 1,
wherein the reaction of step (a) is carried out at 600 °C to 800 °C.

6. The method for producing C2 hydrocarbon compounds and aromatic compounds from methane according to claim 1,
wherein the regeneration temperature of step (c) is maintained the same as the reaction temperature of step (a).

7. The method for producing C2 hydrocarbon compounds and aromatic compounds from methane according to claim 1,
wherein the hydrogen-containing gas of step (c) contains hydrogen in an amount of 10 mol % to 100 mol %.

8. The method for producing C2 hydrocarbon compounds and aromatic compounds from methane according to claim 1,
wherein after step (d), steps (c) and (d) are repeatedly performed.

9. The method for producing C2 hydrocarbon compounds and aromatic compounds from methane according to claim 1,
wherein the C2 hydrocarbon compound comprises at least one selected from the group consisting of ethane, ethylene, and acetylene.

10. The method for producing C2 hydrocarbon compounds and aromatic compounds from methane according to claim 1,
after the step (b), further comprising a step of separately separating the hydrogen, C2 hydrocarbon compounds, and aromatic compounds produced in step (b).

11. The method for producing C2 hydrocarbon compounds and aromatic compounds according to claim 10,
wherein the separated hydrogen is used as the hydrogen-containing gas in step (c).
